(19) United States
(12) Patent Application Publication
(10) Pub. No.: US 2016/0206255 A1
(43) Pub. Date: Jul. 21, 2016

(54) HYBRID PASSIVE/ACTIVE MULTI-LAYER ENERGY DISCRIMINATING PHOTON-COUNTING DETECTOR

(71) Applicants: KABUSHIKI KAISHA TOSHIBA, Tokyo (JP); TOSHIBA MEDICAL SYSTEMS CORPORATION, Tochigi (JP)

(72) Inventors: Daniel GAGNON, Twinsburg, OH (US); Michael Silver, Northbrook, IL (US); Yuexing Zhang, Naperville, IL (US); Gin-Chung Wang, Lincolnshire, IL (US); Yu Zou, Naperville, IL (US)

(73) Assignees: KABUSHIKI KAISHA TOSHIBA, Tokyo (JP); TOSHIBA MEDICAL SYSTEMS CORPORATION, Tochigi (JP)

(21) Appl. No.: 14/599,141

(22) Filed: Jan. 16, 2015

(51) Int. Cl. A61B 6/00; G01T 1/00; A61B 6/03

(52) U.S. Cl. CPC A61B 6/4241; A61B 6/4266; A61B 6/032; G01T 1/00

(57) ABSTRACT

A photon-counting detector apparatus is configured to receive X-rays transmitted from an X-ray source. The photon-counting detector apparatus includes a first photon-counting detector having a first detecting material configured to detect photons using a first set of energy bins. The photon-counting detector apparatus also includes a second photon-counting detector arranged above the first photon-counting detector relative to an incidence direction of the X-rays transmitted from the X-ray source. The second photon-counting detector has a second detecting material configured to detect photons using a second set of energy bins. The first set of energy bins differs from the second set of energy bins.

### BACKGROUND

1. Field

The exemplary embodiments described herein relate to computed tomography (CT) systems. In particular, exemplary embodiments relate to photon-counting detectors.

2. Description of the Related Art

The X-ray beam in most computed tomography (CT) scanners is generally polychromatic. Yet, third-generation CT scanners generate images based upon data according to the energy integration nature of the detectors. These conventional detectors are called energy-integrating detectors and acquire energy integration X-ray data. On the other hand, photon-counting detectors are configured to acquire the spectral nature of the X-ray source, rather than the energy integration nature. To obtain the spectral nature of the transmitted X-ray data, the photon-counting detectors split the X-ray beam into its component energies or spectrum bins and count the number of photons in each of the bins. The use of the spectral nature of the X-ray source in CT is often referred to as spectral CT. Since spectral CT involves the detection of transmitted X-rays at two or more energy levels, spectral CT generally includes dual-energy CT by definition.

Spectral CT is advantageous over conventional CT because spectral CT offers the additional clinical information included in the full spectrum of an X-ray beam. For example, spectral CT facilitates in discriminating tissues, differentiating between tissues containing calcium and tissues containing iodine, and enhancing the detection of smaller vessels. Among other advantages, spectral CT reduces beam-hardening artifacts, and increases accuracy in CT numbers independent of the type of scanner.

Conventional attempts include the use of integrating detectors in implementing spectral CT. One attempt includes dual sources and dual integrating detectors that are placed on the gantry at a predetermined angle with respect to each other for acquiring data as the gantry rotates around a patient. Another attempt includes the combination of a single source that performs kV-switching and a single integrating detector, which is placed on the gantry for acquiring data as the gantry rotates around a patient. Yet another attempt includes a single source and dual integrating detectors that are layered on the gantry for acquiring the data as the gantry rotates around a patient. All of these attempts at spectral CT were not successful in substantially solving issues, such as beam hardening, temporal resolution, noise, poor detector response, poor energy separation, etc., for reconstructing clinically viable images.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a cross-sectional diagram of a combined third-generation and fourth-generation computed tomography apparatus according to one exemplary embodiment;

FIG. 2 illustrates an implementation of a computed tomography system according to one exemplary embodiment;

FIG. 3 is an illustration of photon-counting detectors according to one exemplary embodiment;

FIG. 4 is an illustration of stacked photon-counting detectors according to one exemplary embodiment;

FIG. 5 illustrates a spectrometric detector subsystem of dual-stacked photon detectors according to one exemplary embodiment;

FIG. 6 illustrates a dual-layer of a silicon detector and a CZT detector and the dynamic selection of a detection mode according to one exemplary embodiment; and

FIG. 7 illustrates circuitry that monitors a count rate and feedback according to one exemplary embodiment.

### DETAILED DESCRIPTION

Embodiments herein describe a hybrid passive/active multi-layer energy discriminating photon-counting detector. In some embodiments, a photon-counting detector uses a target layered material to determine the energy level of associated X-ray photons. In some embodiments, a photon-counting detector contains a target having multiple layers of a combined silicon light sensor and scintillator. In some embodiments, a method is described for determining the energy level of X-ray photons by the layer of penetration of the X-ray photons within a target layered material. In some embodiments, a method is described for determining the number of X-ray photons at an associated energy level using a target layered material.

In one embodiment, a photon-counting detector apparatus is configured to receive X-rays transmitted from an X-ray source. The photon-counting detector apparatus includes a first photon-counting detector having a first detecting material configured to detect photons using a first set of energy bins. The photon-counting detector apparatus also includes a second photon-counting detector arranged above the first photon-counting detector relative to an incidence direction of the X-rays transmitted from the X-ray source. The second photon-counting detector has a second detecting material configured to detect photons using a second set of energy bins. The first set of energy bins differs from the second set of energy bins. The first and second detecting materials can be the same or different.

In another embodiment, a CT scanner apparatus includes an X-ray source mounted on a gantry of the CT scanner, and a plurality of photon-counting detectors configured to receive X-rays transmitted from the X-ray source. Each of the plurality of photon-counting detectors includes a first photon-counting detector having a first detecting material configured to detect photons using a first set of energy bins, and a second photon-counting detector above the first photon-counting detector relative to an incidence direction of the X-rays transmitted from the X-ray source, wherein the second photon-counting detector has a second detecting material configured to detect photons using a second set of energy bins. The first set of energy bins differs from the second set of energy bins.

In another embodiment, a dual-stacked photon-counting detector includes a first photon-counting detector having a first detecting material configured to detect photons using a first set of energy bins. The dual-stacked photon-counting detector also includes a second photon-counting detector arranged above the first photon-counting detector relative to an incidence direction of X-rays transmitted from an X-ray source. The second photon-counting detector has a second detecting material configured to detect photons using a second set of energy bins, wherein the first set of energy bins differs from the second set of energy bins. The dual-stacked photon-counting detector also includes circuitry configured to dynamically select, based on a measured count rate, between a first detection mode of photon counting with energy information from the first and second photon-counting detectors, and a second detection mode of photon counting without energy information.

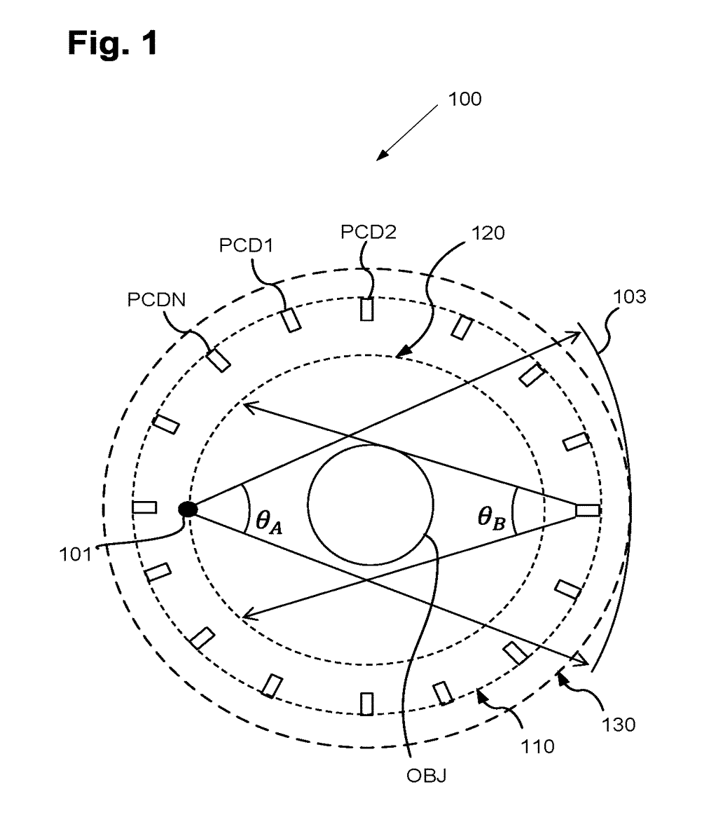

Referring now to the drawings, wherein like reference numerals designate identical or corresponding parts throughout the several views, FIG. 1 is a diagram illustrating an implementation for placing the photon-counting detectors (PCDs) having a predetermined fourth-generation geometry in combination with a detector having a predetermined third-generation geometry in a CT scanner system. The diagram illustrates relative positions among an object OBJ to be scanned, an X-ray source 101, an X-ray detector 103, and the photon-counting detectors PCD1-PCDN, in one exemplary embodiment. For the sake of simplicity, the diagram excludes other components and circuits that may be used in acquiring and processing data as well as reconstructing an image based upon the acquired data. In general, the photon-counting detectors PCD1-PCDN each output a photon count for each predetermined energy bin. In addition to the sparse photon-counting detectors PCD1-PCDN in the fourth-generation geometry, the implementation shown in FIG. 1 includes a detector, such as the detector 103, having a conventional third-generation geometry in the CT scanner system. The detector elements in the detector 103 can be more densely placed along the detector surface than the photon-counting detectors, PCD1-PCDN.

In one implementation, the photon-counting detectors PCD1-PCDN are sparsely placed around the object OBJ in a predetermined geometry such as a circle. For example, the photon-counting detectors PCD1-PCDN are fixedly placed on a predetermined circular component 110 in the gantry 100. In one implementation, the photon-counting detectors PCD1-PCDN are fixedly placed on the circular component 110 at predetermined equidistant positions. In an alternative implementation, the photon-counting detectors PCD1-PCDN are fixedly placed on the circular component 110 at predetermined non-equidistant positions. The circular component 110 remains stationary with respect to the object OBJ and does not rotate during the data acquisition.

Both the X-ray source 101 and the detector 103 rotate around the object OBJ while the photon-counting detectors PCD1-PCDN are stationary with respect to the object OBJ. In one implementation, the X-ray source 101 is mounted on a first rotating portion 120 of the annular frame in the gantry 100 so that the X-ray source 101 projects X-ray radiation with a predetermined source fan beam angle θ_{A} towards the object OBJ while the X-ray source 101 rotates around the object OBJ inside the sparsely placed photon-counting detectors PCD1-PCDN. Furthermore, an additional detector 103 is mounted on a second rotating portion 130 having the third-generation geometry. The rotating portion 130 mounts the detector 103 at a diametrically opposed position from the X-ray source 101 across the object OBJ and rotates outside the stationary circular component 110, on which the photon-counting detectors PCD1-PCDN are fixedly placed in a predetermined sparse manner.

In one implementation, the rotating portions 120 and 130 are integrally constructed as a single component to maintain a fixed angle (such as a 180-degree angle) between the X-ray source 101 and the detector 103 as they rotate about the object OBJ with a different radius. In an optional implementation, the rotating portions 120 and 130 are separate components, but synchronously rotate to maintain the X-ray source 101 and the detector 103 in the fixedly opposed positions at 180-degrees across the object OBJ. Furthermore, the X-ray source 101 optionally travels a helical path as the object is moved in a predetermined direction that is perpendicular to the rotational plane of the rotating portion 120.

As the X-ray source 101 and the detector 103 rotate around the object OBJ, the photon-counting detectors PCD1-PCDN and the detector 103, respectively detect the transmitted X-ray radiation during data acquisition. The photon-counting detectors PCD1-PCDN intermittently detect with a predetermined detector fan beam angle θ_{B} the X-ray radiation that has been transmitted through the object OBJ and each individually output a count value representing a number of photons, for each of predetermined energy bins. On the other hand, the detector elements in the detector 103 continuously detect the X-ray radiation that has been transmitted through the object OBJ and output the detected signals as the detector 103 rotates. In one implementation, the detector 103 has densely placed energy-integrating detectors in predetermined channel and segment directions on the detector surface.

In one implementation, the X-ray source 101, the photon-counting detectors PCD1-PCDN and the detector 103 collectively form three predetermined circular paths that differ in radius. The photon-counting detectors PCD1-PCDN are sparsely placed along a first circular path around the object OBJ while at least one X-ray source 101 rotates along a second circular path around the object OBJ. Further, the detector 103 travels along a third circular path. The above exemplary embodiment illustrates that the third circular path is the largest and outside the first and second circular paths around the object OBJ. Although not illustrated, an alternative embodiment optionally changes the relative relation of the first and second circular paths so that the second circular path for the X-ray source 101 is larger and outside the first circular path of the sparsely placed photon-counting detectors PCD1 through PCDN around the object OBJ. Furthermore, in another alternative embodiment, the X-ray source 101 also optionally travels on the same third circular path as the detector 103. Furthermore, the above alternative embodiments optionally provide a protective rear cover for each of the photon-counting detectors PCD1-PCDN that are irradiated from behind as the X-ray source 101 travels outside the first circular path of the sparsely placed photon-counting detectors PCD1-PCDN.

There are other alternative embodiments for placing the photon-counting detectors having a predetermined fourth-generation geometry in combination with the detector having a predetermined third-generation geometry in the CT scanner. An embodiment includes the X-ray source 101, which is configured to or designed to perform a kV-switching function for emitting X-ray radiation at a predetermined high-level energy and at a predetermined low-level energy.

In general, the photon-counting detectors PCD1-PCDN are sparsely positioned along the circular component 110. Although the photon-counting detectors PCD1-PCDN acquire sparse view projection data, the acquired projection data is sufficient for at least dual-energy (DE) reconstruction with a sparse view reconstruction technique. In addition, the detector 103 also acquires another set of projection data, which is used to generally improve image quality. In the case that the detector 103 consists of energy-integrating detectors with anti-scatter grids, the projection data from the detector 103 is used to correct scatter on the projection data from the photon-counting detectors PCD1-PCDN. In one implementation, the integrating detectors optionally need to be calibrated in view of X-ray transmission through the predetermined circular component 110 and some of the photon-counting detectors PCD1-PCDN. In acquiring the projection data, a sampling on the source trajectory is optionally made sufficiently dense in order to enhance spatial resolution.

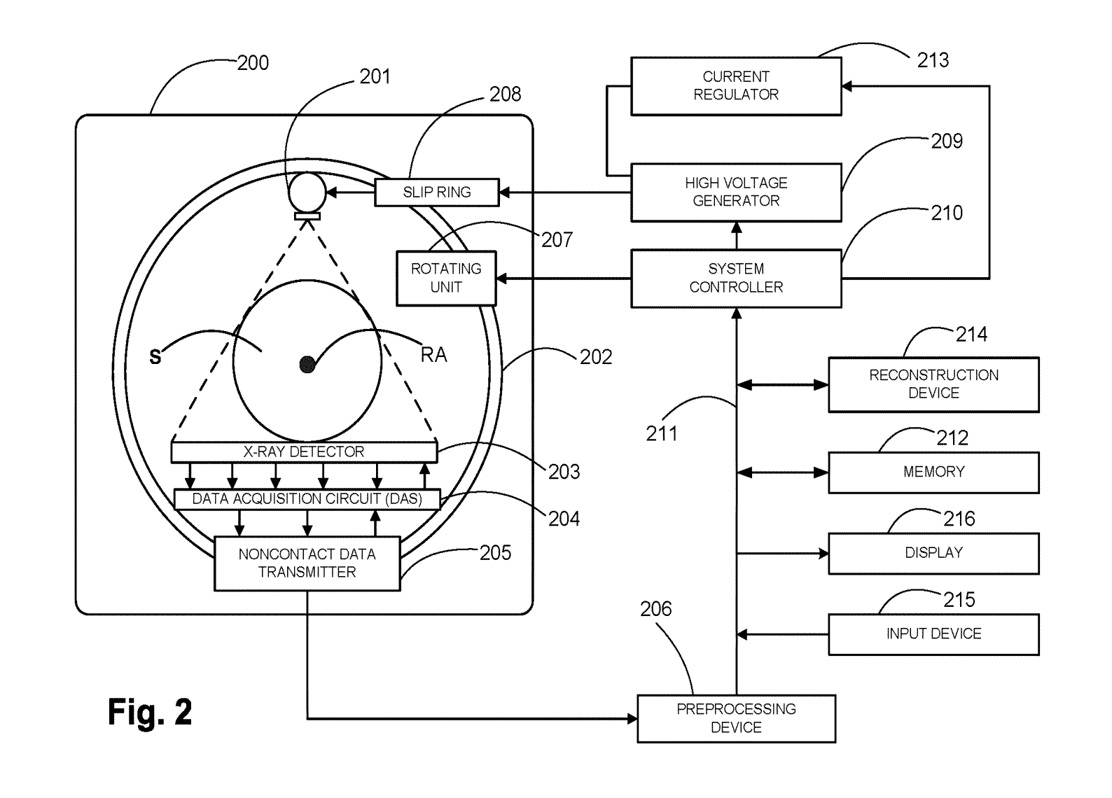

FIG. 2 illustrates an implementation of the radiography gantry 100 of FIG. 1 in a CT apparatus or scanner. As shown in FIG. 2, the radiography gantry 200 is illustrated from a side view and further includes an X-ray tube 201, an annular frame 202, and a multi-row or two-dimensional array type X-ray detector 203. The X-ray tube 201 and X-ray detector 203 are diametrically mounted across a subject S on the annular frame 202, which is rotatably supported around a rotation axis RA. A rotating unit 207 rotates the annular frame 202 at a high speed, such as 0.4 sec/rotation, while the subject S is being moved along the axis RA into or out of the illustrated page.

The multi-slice X-ray CT apparatus further includes a high voltage generator 209 that generates a tube voltage applied to the X-ray tube 201 through a slip ring 208 so that the X-ray tube 201 generates X-rays. The X-rays are emitted towards the subject S, whose cross sectional area is represented by a circle. The X-ray detector 203 is located at an opposite side from the X-ray tube 201 across the subject S for detecting the emitted X-rays that have transmitted through the subject S. The X-ray detector 203 further includes individual detector elements or units.

With continued reference to FIG. 2, the CT apparatus further includes other devices for processing the detected signals from X-ray detector 203. A data acquisition circuit or a Data Acquisition System (DAS) 204 converts a signal output from the X-ray detector 203 for each channel into a voltage signal, amplifies the signal, and further converts the signal into a digital signal. The X-ray detector 203 and the DAS 204 are configured to handle a predetermined total number of projections per rotation (TPPR). Examples of TPPRs include, but are not limited to 900 TPPR, 900-1800 TPPR, and 900-3600 TPPR.

The above-described data is sent to a preprocessing device 206, which is housed in a console outside the radiography gantry 200 through a non-contact data transmitter 205. The preprocessing device 206 performs certain corrections, such as sensitivity correction on the raw data. A memory 212 stores the resultant data, which is also called projection data at a stage immediately before reconstruction processing. The memory 212 is connected to a system controller 210 through a data/control bus 211, together with a reconstruction device 214, input device 215, and display 216.

The detectors are rotated and/or fixed with respect to the patient among various generations of the CT scanner systems. The above-described CT system is an example of a combined third-generation geometry and fourth-generation geometry system. In the third-generation system, the X-ray tube 201 and the X-ray detector 203 are diametrically mounted on the annular frame 202 and are rotated around the subject S as the annular frame 202 is rotated about the rotation axis RA. In the fourth-generation geometry system, the detectors are fixedly placed around the patient and an X-ray tube rotates around the patient.

In an alternative embodiment, the radiography gantry 200 has multiple detectors arranged on the annular frame 202, which is supported by a C-arm and a stand.

As discussed above, photon-counting detectors are used for spectral X-ray and CT applications. One method to encode the energy of a detected photon is to measure the effect of the interaction of the photon in a material in an active energy-discrimination process. A direct or an indirect class of detector can be used. In an indirect detector, the photon interaction will create light that is measured with a light sensor. In a direct detector, the photon directly creates a charge in the material that is collected and fed to a specifically designed electrical circuit.

A method of obtaining energy information is through a passive energy-discrimination process. Interaction of photons with matter is stochastic in nature, and parameters of the interaction depend on the energy of the photon and the nature of the material, such as its electron density and effective atomic number. A configuration of two or more stacked detectors has the ability to extract information on the interactions of the photons at each detector in the stack. A stacked configuration of photon-counting detectors provides information of a depth of interaction of X-ray photons, which is energy dependent.

When designing a configuration of multiple-stacked photon-counting detectors, the thickness of each detector should be set such that a proper ratio of interaction between the detectors is obtained. The detector layer closer to the entrance plane of the radiation should be thinner than a detector layer farther from the entrance plane in order to capture the same or nearly the same amount of photons. In addition, the multiple-stacked photon-counting detectors need to have parameters such that an effective spectrum of energy levels is collected and counted for a broad energy beam or flux. Also, photons from a lower energy portion of the spectrum are more likely to be detected in the closer detector(s) of radiation penetration, and photons with more energy are detected in the deeper detector(s). As a result, dual or multi-stacked detectors can passively encode an energy distribution of an incoming photon beam.

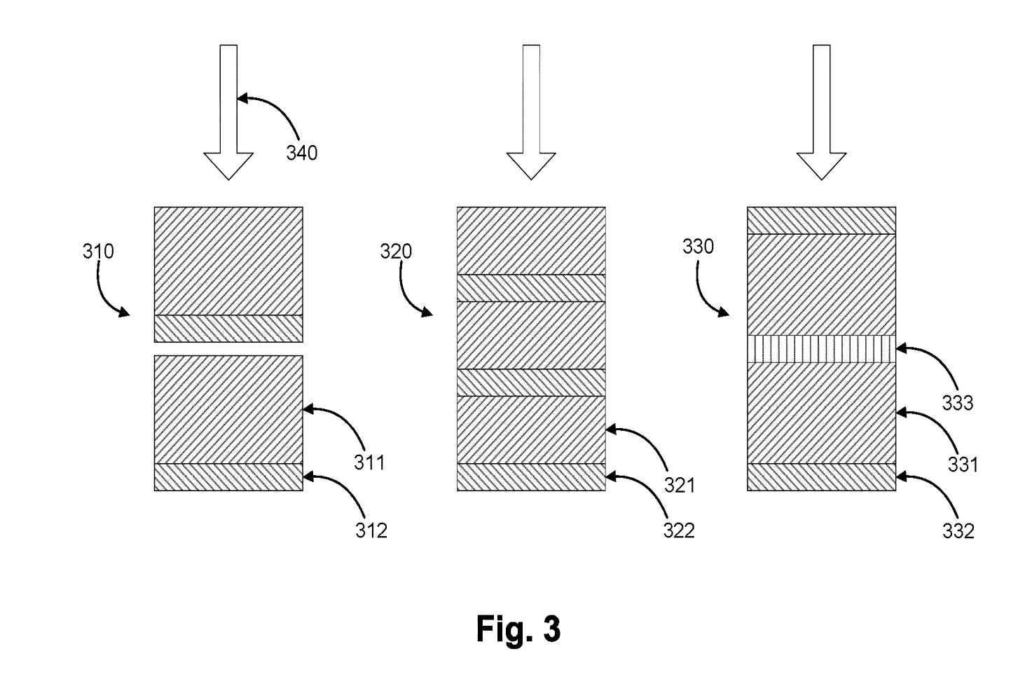

FIG. 3 illustrates three embodiments of photon-counting detector apparatuses, each of which has a layered or stacked configuration of photon-counting detectors. The depth of interaction of the X-ray photons is energy-dependent. The layered configuration of detectors also uses silicon electronics, which have a low attenuation to X-rays. The photon-counting detector apparatuses in FIG. 3 include dual-stacked photon-counting detectors 310, a stacked configuration of more than two photon-counting detectors 320, and a dual-stacked configuration with a photon-counting detector 330.

The dual-stacked detector apparatus 310 has a double-stacked layer of a scintillator 311 and a light sensor 312, such as a SiPM. X-rays 340 enter the dual-stacked detector apparatus 310 at the scintillator 311 layer.

The multi-stacked detector apparatus 320 is stacked with three or more layers of a combined scintillator 321 and a light sensor 322.

The single photon-counting detector 330 has a dual-layer of two mirrored combinations of scintillator 331 and light sensor 332. As a result, the two stacked detectors share a common detector layer 333. Each light sensor layer and each scintillator layer of FIG. 3 can have a different thickness. The multiple scintillators can also be formed of different scintillation materials.

Another example of a target material for a photon-counting detector is cadmium zinc telluride (CZT). The interaction of the photons with the semiconductor matter plays an important role in the active energy discrimination process, as well as the passive energy discrimination process. For a direct conversion system, the detector needs to be thick enough to capture most of the photons, but thin enough to speed up the charge collection.

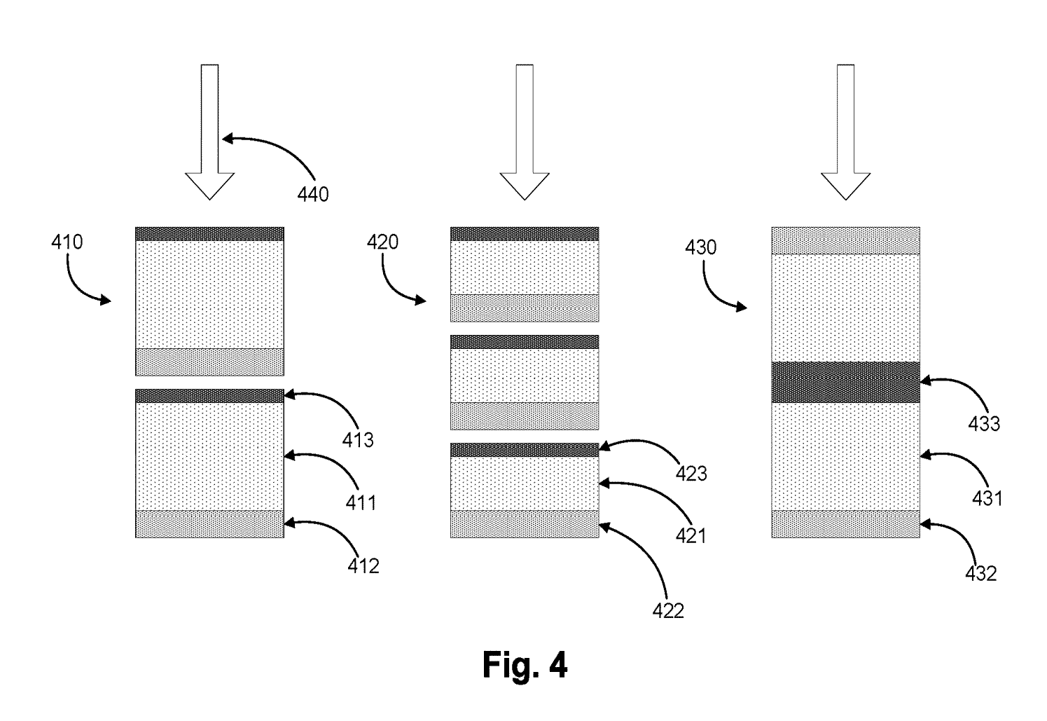

FIG. 4 illustrates three embodiments of stacked photon-counting detector apparatuses in which a combination of passive and active energy discrimination detection features are combined into a layered configuration of multiple photon-counting detectors.

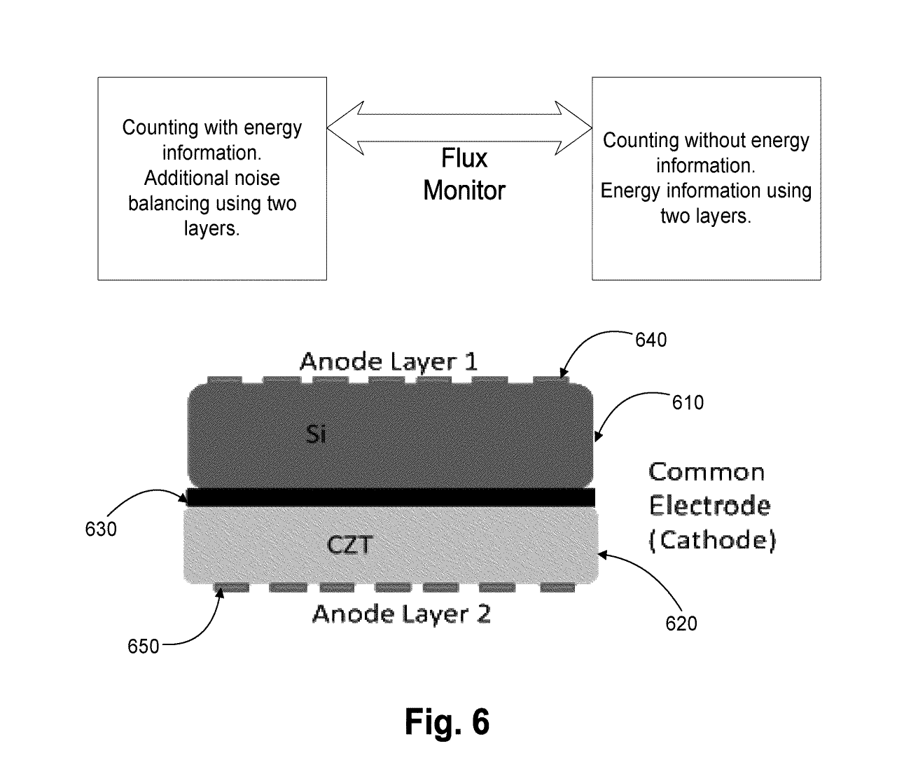

A dual-stacked configuration of photon-counting detectors 410 includes a bulk semiconductor material 411 in combination with a semiconductor compound. One embodiment includes a combination of CZT and CdTe. However, other materials and an associated semiconductor compound are used in embodiments described herein. An anode layer 412 is located at a lower surface of the semiconductor material 411. Anode layer 412 also includes application-specific integrated circuitry to detect and convert an electrical current pulse into respective energy bins according to their energy levels (see FIG. 6).

A cathode layer 413 is located at an upper surface of the semiconductor material 411. X-ray radiation 440 enters the dual-stacked semiconductor photon detectors 410 at the cathode layer 413. In the dual-stacked detector configuration 410, two semiconductor photon-counting detectors are stacked to provide encoding information from the photon spectrum according to the spectrum energy and the characteristics of each semiconductor photon-counting detector.

A multi-layered photon-counting detector configuration 420 containing three or more detectors is illustrated in FIG. 4. Each detector contains a bulk semiconductor material 421 and a semiconductor compound, such as CZT/CdTe, along with a lower anode layer 422 and an upper cathode layer 423.

A dual-stacked detector configuration with a shared cathode 430 is also illustrated in FIG. 4. The combined configuration 430 includes a bulk semiconductor material 431, an anode layer 432 at either end, and a shared cathode layer 433. X-ray radiation enters the combined detector configuration at the upper anode layer 432.

In addition to the passive energy discrimination processing, embodiments described herein analyze the signal from each stacked detector and record the energy information. The detector response for ballistic deficit, pile-up, and charge sharing are determined from the recorded information. The detector apparatuses illustrated in FIG. 4 combine the attributes of multiple stacked detectors and a combination of active and passive energy discrimination detection features into one detector system.

Several advantages result from the above-described stacked detector system. Segmenting the overall thickness of detectors into multiple layers increases the total count rate since each detector can be designed to operate at or near the maximum counting rate. The counting rate is simulated by simulation in advance and the thickness of each layer is defined based on the simulated counting rate. The maximum counting rate will depend on the characteristics of the semiconductor material and/or the electronic circuitry. Another advantage is that each detector can count faster than one single, thicker system, since the thickness of the semiconductor material influences the speed at which the charges from a photon interaction are collected. Also, if the energy discriminating rate of the stacked detector system is exceeded, the readout circuitry and method could be converted to a simpler counting system that does not record energy information. If the rate exceeds the maximum capability of the system, the system could dynamically switch from a spectrometric mode to a counting mode. In this case, all the photons would still be kept and would still carry some energy information from the passive segregation of the multiple-stacked detectors.

Another variation of embodiments described herein is a progressive change of the integration time of the energy discriminating detector. This creates multiple quality levels of energy discrimination, in contrast to the binary state of recording or not recording energy information, as described above. In one embodiment, multiple flux rate thresholds can be used to trigger corresponding integration time changes for each detector.

Another advantage of embodiments described herein includes having more information from spectrometric measurements of the multiple-stacked detectors in a nominal mode than a single layer of a same or similar detector. The multiple-stacked detector system has statistical samples from several realizations of the incoming radiation beam. Another advantage of multiple-stacked semiconductor photon-counting detectors is a very thin electrode with essentially no effect on the incoming photon beam. In addition, if the first detector is silicon-based, a lower density in a z-axis direction offers a preferential detection of lower energy of the energy spectrum, which offers a wider range of thicknesses for overall optimization.

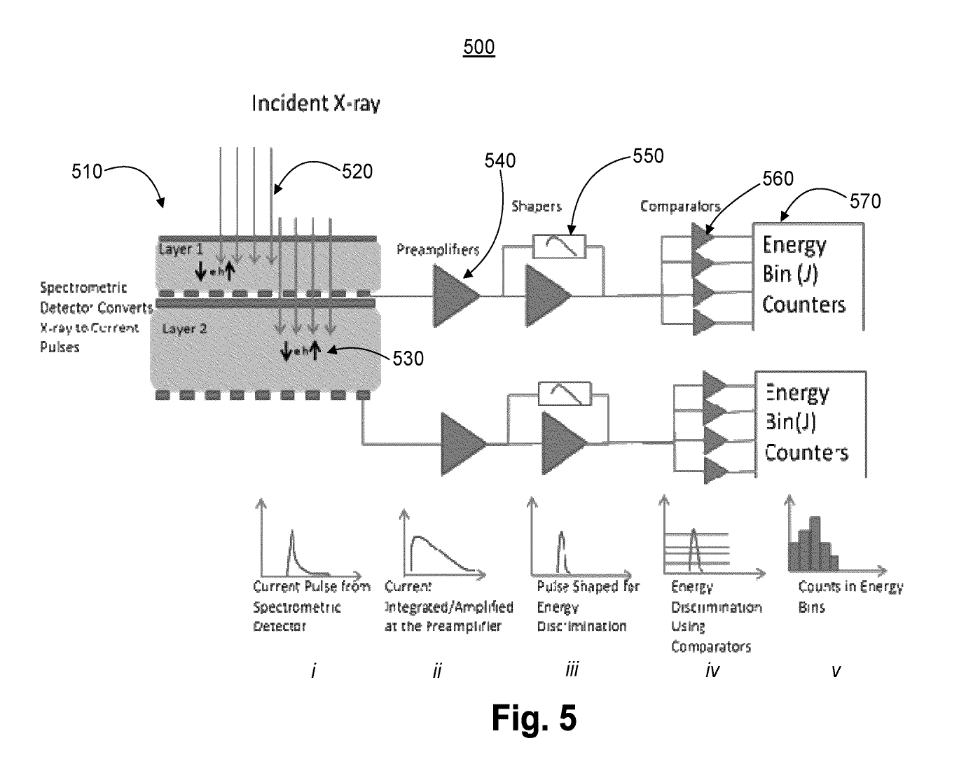

FIG. 5 illustrates a spectrometric detector subsystem 500 of dual-stacked photon detectors 510 that converts each incident X-ray photon 520 into an electrical signal. The energy information of each X-ray photon 520 is preserved. Each signal is conditioned and processed through integrated circuitry, such as application-specific integrated circuitry, to obtain the energy information of each X-ray photon 520 and to classify the obtained energy information into energy bins. As illustrated in FIG. 5, the dual-stacked photon detectors 510 convert the incoming X-ray photons 520 into electron (e) and hole (h) pairs 530. The generated electrons and holes are swept by an internal electric field and collected by anode and cathode electrodes, respectively. The electron and hole movements induce current pulses, as illustrated by the current pulse i. The current pulses are amplified and/or integrated by a preamplifier 540, as illustrated by the current pulse ii. The current pulses are filtered and/or shaped for energy discrimination by shapers 550, as illustrated by the current pulse iii, and sent to an array of comparators 560, as illustrated by the current pulse iv. The pre-determined thresholds of the comparators register the counts of the X-rays into their corresponding energy windows into energy bin counters 570, as illustrated by the energy bin counts v.

FIG. 6 illustrates a silicon detector 610 and a CZT detector 620 that share a common cathode electrode 630. An anode layer 640 is located at the outside surface of the silicon detector 610, and an anode layer 650 is located at the outside surface of the CZT detector 620. One advantage of using a common electrode between two mirrored detectors is a reduction in the depth of a dependent energy response, usually for an electron. When the X-ray is incident from a cathode side, small anode pixels reduce this effect. However, if both electrons and holes can travel to a corresponding electrode in a time that is shorter than the shaping time of the electronics, there is no such depth-dependent energy response problem. Stated another way, if the upper detector uses a material such as silicon (a material in which both carriers move at a fast pace), a common electrode can be used to reduce the complexity of packaging, thereby reducing costs. A common electrode will also reduce the trace capacitance, thereby improving the electronic noise. When a common cathode is not used, there is additional capacitance from the anode layer to the cathode layer. The dual-stacked photon-counting detector includes circuitry (described below) configured to dynamically select, based on a measured count rate, between a first detection mode of photon counting with energy information from the first and second photon-counting detectors, and a second detection mode of photon counting without energy information.

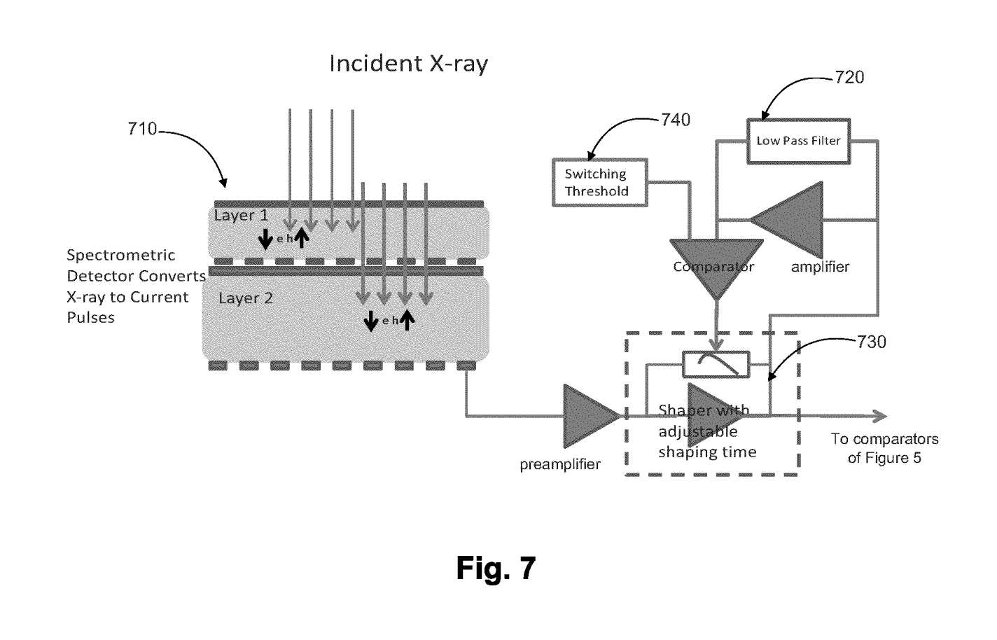

FIG. 7 illustrates one embodiment of circuitry that monitors the count rate and adjusts the shaping time of the shaper of a dual-stacked photon detector 710, so as to effectively switch detection modes. As shown in FIG. 7, the circuitry includes a low-pass filter 720, which samples the baseline (DC value) of the signal after passing through a shaper 730, which is a good indicator of the flux level. Note that the DC value increases when pulses start piling up on top of each other, which reflects an increase in the count rate. When the baseline reaches a predetermined switching threshold 740, the shaping time of the shaper is shortened so as to result in the counting-only mode shown in FIG. 6 (without recording energy information). Alternatively, the shaping time can be incrementally changed based on the detected flux level so that a plurality of detection modes can be used.

In another embodiment, the system dynamically selects a detection mode. There is sometimes a tradeoff between the shaping time of the filter and the energy information of each incident X-ray event. At a high flux rate, a shorter shaping time might be preferred to reduce resultant pile-up. However, a shorter shaping time can lead to more electronic noise due to a wider electronic bandwidth and more statistical noise due to a ballistic deficit. The additional noise significantly decreases the content of energy information of each X-ray when the shaping time is shorter than the detector response time. Thus, a dual-stacked detector design enables a mode in which the shaping time can be shortened, in which only thresholding information is preserved. The energy information can still be retrieved from the energy-dependent absorption of the two detectors. The diagram of FIG. 7 illustrates the dynamic selection of a detection mode.

In another embodiment, the count rate of each layer can be monitored (and the detection mode changed) separately using circuitry similar to that shown in FIG. 7.

A system with two modes, as described above can handle a wider range of flux yet still retain some energy information of each X-ray, which provides an advantage over systems that switch between photon-counting and energy-integrating modes.

Another embodiment is now described to illustrate how to use energy bin counts from two or more detector layers. Assume there are K layers and Jₖ measurements for layer k for each ray path. The measured signal can be expressed as,

$${I}_{kj} = \int d {ES}_{k} ( E ) {R}_{kj} ( E ) exp [ - {\sum }_{n = 1}^{N} {μ}_{n} ( E ) {L}_{n} ] , \\ j = 1 , 2 , 3 , … {J}_{k} ; k = 1 , 2 , 3 , … K ,$$

where Rₖⱼ(E) is the detector response for layer k and measurement j at photon energy E, μₙ(E) is the linear attenuation of basis n, Lₙ is the length of basis n in the ray path, and Sₖ(E) is the spectrum for layer k in an air scan. If the source spectrum is S₀(E), the air spectrum for detector layer k can be expressed as

Sₖ(E)=Sₖ₋₁(E)exp[—μₖ₋₁(E)Δₖ₋₁],

where μₖ(E) is the linear attenuation of layer k and Δₖ is the corresponding thickness. Note that Δ₀=0. The air calibrated data can be written as,

gₖⱼ=ln Iₖⱼ^{(α)}−ln Iₖⱼ,

where

Iₖⱼ^{(α)}=∫dESₖ(E)Rₖⱼ(E), j=1,2,3, . . . Jₖ; k=1,2,3, . . . K.

Assuming the variance of gₖⱼ is σₖⱼ², there is a weighted least-squares cost function,

$$ψ ( L ) = {\sum }_{k = 1}^{K} {\sum }_{j = 1}^{{J}_{k}} \frac{1}{{σ}_{kj}^{2}} {( {g}_{kj} ( L ) - {g}_{kj}^{( M )} )}^{2} ,$$

where gₖⱼ^{(M)} is the measured air calibrated data with variance σₖⱼ², gₖⱼ(L) is the forward projection data, and vector L=(L₁, L₂, L₃, . . . L_{N})^{T} contains the material lengths. By minimizing the cost function, the material lengths Lₙ can be found.

Different energy thresholds can be applied to each of the stacked photon-counting detectors. In one embodiment, each of the stacked photon-counting detectors has a different thickness. Another embodiment includes applying a lower energy threshold to photon-counting detectors near the incident X-ray photons, and applying a higher energy threshold to photon-counting detectors farther away from the incident X-ray photons.

While certain implementations have been described, these implementations have been presented by way of example only, and are not intended to limit the scope of the disclosure. The novel devices, systems and methods described herein may be embodied in a variety of other forms. Furthermore, various omissions, substitutions, and changes in the form of the devices, systems, and methods described herein may be made without departing from the spirit of the disclosures. The accompanying claims and their equivalents are intended to cover such forms or modifications as would fall within the scope and spirit of the disclosures.

1. A photon-counting detector apparatus configured to receive X-rays transmitted from an X-ray source, the photon-counting detector apparatus comprising:
a first photon-counting detector having a first detecting material configured to detect photons using a first set of energy bins; and
a second photon-counting detector arranged above the first photon-counting detector relative to an incidence direction of the X-rays transmitted from the X-ray source, the second photon-counting detector having a second detecting material configured to detect photons using a second set of energy bins, wherein the first set of energy bins differs from the second set of energy bins.

2. The photon-counting detector apparatus of claim 1, wherein a thickness of the first detecting material differs from a thickness of the second detecting material.

3. The photon-counting detector apparatus of claim 1, wherein the first detecting material and the second detecting material are semiconductor compounds.

4. The photon-counting detector apparatus of claim 3, further comprising a cathode adjacent to a first surface of the first detecting material and a first surface of the second detecting material, and an anode adjacent to a second surface of the first detecting material and a second surface of the second detecting material.

5. The photon-counting detector apparatus of claim 3, further comprising a shared cathode between the first and the second photon-counting detectors of the photon-counting detector apparatus.

6. The photon-counting detector apparatus of claim 1, further comprising a third photon-counting detector arranged above the second photon-counting detector relative to the X-rays transmitted from the X-ray source, and configured to receive and count photons using a third set of energy bins.

7. The photon-counting detector apparatus of claim 1, wherein the first and the second photon-counting detectors have circuitry configured to determine an energy of each received photon.

8. The photon-counting detector apparatus of claim 1, further comprising one or more comparators configured to count a number of received photons at each photon energy.

9. The photon-counting detector apparatus of claim 1, wherein the first detecting material and the second detecting material are a same material.

10. The photon-counting detector apparatus of claim 1, wherein the first detecting material and the second detecting material are different materials.

11. A computed tomography (CT) scanner apparatus, comprising:
an X-ray source mounted on a gantry of the CT scanner; and
a plurality of photon-counting detectors configured to receive X-rays transmitted from the X-ray source, each of the plurality of photon-counting detectors including
a first photon-counting detector having a first detecting material configured to detect photons using a first set of energy bins; and
a second photon-counting detector arranged above the first photon-counting detector relative to an incidence direction of the X-rays transmitted from the X-ray source, the second photon-counting detector having a second detecting material configured to detect photons using a second set of energy bins, wherein the first set of energy bins differs from the second set of energy bins.

12. The CT scanner apparatus of claim 11, further comprising circuitry configured to determine an energy of each received photon.

13. The CT scanner apparatus of claim 11, further comprising one or more comparators configured to count a number of received photons at each photon energy.

14. The CT scanner apparatus of claim 11, wherein
the X-ray source is configured to rotate about a patient table;
the plurality of photon-counting detectors are stationary; and
the CT scanner apparatus further comprises a detector array including a plurality of X-ray detectors configured to rotate about the patient table in synchronization with the rotating X-ray source.

15. A dual-stacked photon-counting detector, comprising:
a first photon-counting detector having a first detecting material configured to detect photons using a first set of energy bins;
a second photon-counting detector arranged above the first photon-counting detector relative to an incidence direction of X-rays transmitted from an X-ray source, the second photon-counting detector having a second detecting material configured to detect photons using a second set of energy bins, wherein the first set of energy bins differs from the second set of energy bins; and
circuitry configured to dynamically select, based on a measured count rate, between a first detection mode of photon counting with energy information from the first and second photon-counting detectors, and a second detection mode of photon counting without energy information.

16. The dual-stacked photon-counting detector of claim 15, wherein the circuitry is configured to select the first detection mode when a flux rate is high.

17. The dual-stacked photon-counting detector of claim 15, wherein the circuitry is configured to retrieve energy information using both layers of the dual-stacked photon-counting detector in the second detection mode.

18. The dual-stacked photon-counting detector of claim 15, wherein the circuitry is further configured to dynamically select between the first and second detector modes based on at least one count rate threshold, and is configured to alter an integration time of energy discriminating detectors.
